(19) United States
(12) Patent Application Publication
(10) Pub. No.: US 2011/0159304 A1
(43) Pub. Date: Jun. 30, 2011

(54) ANTI-SULPHATE REDUCING BACTERIA COMPOSITION COMPRISING 1,2-BENZISOTHIAZOL-3(2H)-ONE, IRGASAN, BENZYL-2-BROMOACETATE, 2,2-DIBROMO-2-CYANOACETAMIDE, AND 2-BROMO-2-NITROPROPAN-1,3-DIOL

(75) Inventors: Hong Seok Song, Gunpo (KR); Young Geun Kim, Ansan (KR); Keun Chang Ryu, Suwon (KR); Young Min Baek, Hwaseong (KR); Young Hyo Chang, Daejeon (KR)

(73) Assignee: KOREA GAS CORPORATION, Ansan (KR)

(21) Appl. No.: 12/792,342

(22) Filed: Jun. 2, 2010

(30) Foreign Application Priority Data
Dec. 28, 2009 (KR) 10-2009-132072
Dec. 28, 2009 (KR) 10-2009-132079
Dec. 28, 2009 (KR) 10-2009-132084
Dec. 28, 2009 (KR) 10-2009-132093
Dec. 28, 2009 (KR) 10-2009-132095

(51) Int. Cl. A61K 31/428; C07D 275/04; C07C 43/257; A61K 31/075; C07C 69/00; A61K 31/185; C07C 249/00; A61K 31/16; C07C 205/00; A61K 31/045; B32B 15/04

(52) U.S. Cl. 428/457; 548/209; 514/373; 568/635; 514/717; 560/129; 514/578; 558/302; 514/626; 568/712; 514/727

(57) ABSTRACT

Disclosed are a composition for inhibiting a proliferation of sulphate reducing bacteria comprising at least one of 1,2-benzisothiazol-3(2H)-one, irgasan, benzyl-2-bromoacetate, 2,2-dibromo-2-cyanoacetamide, and 2-bromo-2-nitropropan-1,3-diol as effective ingredients; a method for inhibiting the proliferation of sulphate reducing bacteria comprising the step of including a sufficient amount for inhibiting the proliferation of sulphate reducing bacteria of at least one of 1,2-benzisothiazol-3(2H)-one, irgasan, benzyl-2-bromoacetate, 2,2-dibromo-2-cyanoacetamide, and 2-bromo-2-nitropropan-1,3-diol in a corrosion sensitive material or degradation sensitive material; a sheet comprising the composition; and a steel plate to which the composition is applied.

### CROSS-REFERENCE TO RELATED APPLICATION

This application claims under 35 U.S.C. §119(a) the benefit of Korean Application Nos. 10-2009-132072, 10-2009-132079, 10-2009-132084, 10-2009-132093, and 10-2009-132095 filed Dec. 28, 2009, the entire contents of which are incorporated herein by reference.

### TECHNICAL FIELD

The present invention relates to a composition for inhibiting a proliferation of sulphate reducing bacteria comprising 1,2-benzisothiazol-3(2H)-one, irgasan, benzyl-2-bromoacetate, 2,2-dibromo-2-cyanoacetamide, and 2-bromo-2-nitropropan-1,3-diol as effective components; a method for inhibiting the proliferation of sulphate reducing bacteria comprising the step of including a sufficient amount for inhibiting the proliferation of sulphate reducing bacteria of 1,2-benzisothiazol-3(2H)-one, irgasan, benzyl-2-bromoacetate, 2,2-dibromo-2-cyanoacetamide, and 2-bromo-2-nitropropan-1,3-diol in a corrosion sensitive material or degradation sensitive material; a sheet comprising the composition; and a steel plate to which the composition is applied.

More specifically, the present invention relates to a composition comprising 1,2-benzisothiazol-3(2H)-one, irgasan, benzyl-2-bromoacetate, 2,2-dibromo-2-cyanoacetamide, and 2-bromo-2-nitropropan-1,3-diol which inhibits a proliferation of sulphate reducing bacteria on easily corroded or degraded metal, concrete, mortar and other surfaces.

### BACKGROUND ART

Coatings are generally applied to underground pipelines (gas pipeline, water pipeline, oil pipeline, etc.) to prevent the pipeline from corrosion in the soil environment. To the girth weld of the pipeline, after welding, may also be applied epoxy or polyurethane and the like in the general paint form. However, if the coating material applied to mainline pipelines is polyolefin, the coating material having similar properties is also applied to the girth weld. Usually applied is the coating material consisting of a adhesive sheet bonded to the outer surface of the welded pipeline and an insulating polymer protecting the sheet from outside thereof as a physical backing layer. The typical coating materials applied in such a way include a heat shrinkable sheet which is applied using a flame of torch and a tape which is rolled up directly at room temperature.

In a case that bonding of the coating material is maintained well, the corrosive elements present in the surrounding soil environment cannot be directly contacted with the pipeline bare surface. However, if coating material is applied via an inappropriate pretreatment or has a low quality, the material may be detached out of the pipeline or be wrinkled by soil stress over time. While the corrosive elements are penetrated into such pocket, the protective cathodic current applied from outside cannot be provided sufficiently through the small detached channel, and thus, it is hard to prevent the corrosion. In particular, if the sulphate reducing bacteria, which is an anaerobic microbe to promote steel's corrosion significantly, lives in the surrounding environment leading to metabolizes at the site, the rapid corrosion by the bacteria corrosion (microbial corrosion) may proceeds.

In the soil environment, most of microbial corrosions are related with the anaerobic sulphate reducing bacteria (SRB). This bacteria inhabits in the soil having a high water content, clay content, organic matter content and the like, and is responsible for a very serious corrosion of the buried pipeline.

SRB utilizes sulphate as a terminal electron acceptor in one step of complex metabolic activity in view of ecological properties. That is, the sulphate is reduced by SRB to form sulfide (S²⁻). This sulfide itself is a very corrosive, and reacts with hydrogen ion to form hydrogen sulfide (H₂S) that has also the great corrosiveness, or binds to the surrounding iron ion (Fe²⁺) to form a black iron sulfide (FeS) film on the surface of pipeline as a corrosion product. Therefore, several deep corrosion pits or relatively uniform striations are formed in the site where the microbial corrosion is developed and the surface thereof is completely covered with black films. The conventional method for inhibiting the corrosion under closed circulation water system is to change water environment (pH adjustment, corrosion inhibiting agent and bactericide input, deoxidizer input, etc.). In case of internal and external protection of long-distance ground pipelines, it is difficult to change both inside and outside of the pipelines, and thus, coating is mainly applied thereto. To protect the corrosion of the site having coating being damaged, a cathodic protection (Sacrificial Anode Method and Impressed Current Method) is used in combination with the coating. An cathodic protection is a method for preventing the corrosion by providing an excess of electrons so as to inhibit the corrosion reaction (Fe→Fe²⁺2e⁻) in the pipeline surface of coating damaged portion promoting only reduction reactions such as the reverse reaction of the reaction mentioned above (Fe←Fe²⁺2e⁻) or the reduction reaction of oxygen (2H₂O+O₂+4e⁻→4OH⁻) may occur. Under the condition that the sufficient reduction reactions occur, it is reported that the corrosion reaction does not almost proceed and microbial corrosion is also inhibited significantly.

However, even though the cathodic protection is applied, if the damaged portion is formed in such a way that the coating having the insulating external sheet is detached from the pipelines, the protective current cannot reach effectively the pipeline surface underlying the detached coating so called shielding effect. That is, while the protective current does not reach the pipeline surface through the insulating coating sheet, the current flows insufficiently only through the electrolyte between the pipeline and the detached coating layer. In such a case, the sufficient reduction reaction is difficult to develop in the pipeline surface, and thus, in addition to a general type of corrosion, the microbial corrosion can also proceed rapidly under conditions favorable for microbe inhabitation.

In constructing a pipeline, applying a heat shrinkable sheet or tape to a girth weld can lead to deterioration in coating performance due to poor surface treatment or insufficient heating. Furthermore, the soil subsidence after burying the pipe, applies shear stress to coating whereby coating defect, by which the coating droops to around 6 o'clock direction, can arise. Also, if the soil environment surrounding coating defect shows the condition favorable for inhabitance of sulphate reducing bacteria, metabolites produced by the sulphate reducing bacteria proliferated inside the defect cause the pipelines to be corroded at rapid rate. Therefore, such problems need to be solved.

The above information disclosed in this Background Art section is only for enhancement of understanding of the background of the invention and therefore it may contain information that does not form the prior art that is already known in this country to a person of ordinary skill in the art.

### SUMMARY OF THE INVENTION

The present invention reveals that 1,2-benzisothiazol-3(2H)-one, irgasan, benzyl-2-bromoacetate(beanzyl-2-bromoacetate), 2,2-dibromo-2-cyanoacetamide(2,2-dibromo-2-cyanoacetamide), and/or 2-bromo-2-nitropropan-1,3-diol(2-bromo-2-nitropropan-1,3-diol) inhibit the proliferation of sulphate reducing bacteria, and have an excellent antibacterial effect even in applying thermal shock. In addition, the present invention solved the aforementioned problems by providing a sheet comprising the composition including an effective amount of at least one of 1,2-benzisothiazol-3(2H)-one, irgasan, benzyl-2-bromoacetate, 2,2-dibromo-2-cyanoacetamide, and 2-bromo-2-nitropropan-1,3-diol, and the steel plate to which the antibacterial composition is applied.

The above features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

The above and other objects and features of the present invention will become apparent from the following description of embodiments, given in conjunction with the accompanying drawings, in which:

FIGS. 1a to 1e are photographies of the results of the antibacterial assay of 1,2-benzisothiazol-3(2H)-one, irgasan, benzyl-2-bromoacetate, 2,2-dibromo-2-cyanoacetamide, and 2-bromo-2-nitropropan-1,3-diol;

FIG. 2 is a photography of the results of the antibacterial assay of the control compound;

FIGS. 3a to 3e are photographies of the results of the antibacterial assay of 1,2-benzisothiazol-3(2H)-one, irgasan, benzyl-2-bromoacetate, 2,2-dibromo-2-cyanoacetamide, and 2-bromo-2-nitropropan-1,3-diol before and after thermal shock;

FIG. 4 is a photography of the results of the antibacterial assay of the control compound before and after thermal shock;

FIGS. 5a to 5d are photographies of the test results of antibacterial activity of 1,2-benzisothiazol-3(2H)-one, irgasan, benzyl-2-bromoacetate, 2,2-dibromo-2-cyanoacetamide, and 2-bromo-2-nitropropan-1,3-diol in an adhesive at varying concentrations;

FIGS. 6a to 6e are photographies of the test results of antibacterial activity of 1,2-benzisothiazol-3(2H)-one, irgasan, benzyl-2-bromoacetate, 2,2-dibromo-2-cyanoacetamide, and 2-bromo-2-nitropropan-1,3-diol in a primer at varying concentrations;

FIG. 7 shows the results of peeling strength experiment, wherein the dotted line indicates the requirement value in European standard EN12068;

FIG. 8 shows the results of cathodic disbondment experiment, wherein the dotted line indicates the requirement value in European standard EN12068; and

FIG. 9 shows the results of shear strength experiment, wherein the dotted line indicates the requirement value in European standard EN12068.

### DETAILED DESCRIPTION OF THE EMBODIMENTS

Hereinafter, embodiments of the present invention will be described in detail with the accompanying drawings.

The present invention relates to a composition for inhibiting a proliferation of sulphate reducing bacteria comprising 1,2-benzisothiazol-3(2H)-one, irgasan, benzyl-2-bromoacetate, 2,2-dibromo-2-cyanoacetamide, and 2-bromo-2-nitropropan-1,3-diol as effective components.

The abovementioned 1,2-benzisothiazol-3(2H)-one has a structure of a chemical formula as shown below:

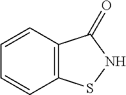

Further, the irgasan has a structure of a chemical formula as shown below:

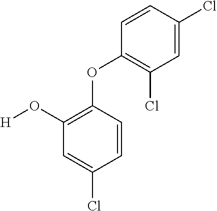

Furthermore, the benzyl-2-bromoacetate has a structure of a chemical formula as shown below:

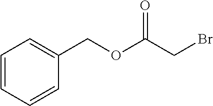

Furthermore, the 2,2-dibromo-2-cyanoacetamide has a structure of a chemical formula as shown below:

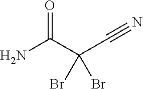

Furthermore, the 2-bromo-2-nitropropan-1,3-diol has a structure of a chemical formula as shown below:

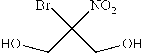

The composition for inhibiting a proliferation of sulphate reducing bacteria according to the present invention can further comprise a binder wherein such binder is a commonly used one.

Furthermore, the present invention relates to a method for inhibiting the proliferation of sulphate reducing bacteria in corrosion sensitive material or degradation sensitive material by using a sufficient amount for inhibiting the proliferation of sulphate reducing bacteria of 1,2-benzisothiazol-3(2H)-one, irgasan, benzyl-2-bromoacetate, 2,2-dibromo-2-cyanoacetamide, and 2-bromo-2-nitropropan-1,3-diol. Such corrosion sensitive material can be a metal, and specifically metal may include, but is not limited to, carbon steel, stainless steel, aluminum, aluminum alloy, copper, copper alloy, titanium, titanium alloy, nickel or nickel alloy and the like.

Such degradation sensitive material may include, but is not limited to, concrete, reinforced concrete or cement.

The present invention is directed to a sheet containing the composition for inhibiting a proliferation of sulphate reducing bacteria comprising 1,2-benzisothiazol-3(2H)-one, irgasan, benzyl-2-bromoacetate, 2,2-dibromo-2-cyanoacetamide, and 2-bromo-2-nitropropan-1,3-diol.

Such sheet can be a sheet applied on a corrosion sensitive material or a degradation sensitive material, and particularly at least one sheet selected from the group consisting of a heat shrinkable sheet, a adhesive sheet, an insulating polymer sheet and a plastic sheet.

The present invention is also directed to a steel plate to which the composition for inhibiting a proliferation of sulphate reducing bacteria is applied, wherein the composition comprises 1,2-benzisothiazol-3(2H)-one, irgasan, benzyl-2-bromoacetate, 2,2-dibromo-2-cyanoacetamide, and 2-bromo-2-nitropropan-1,3-diol.

The composition of the present invention has the advantage as follows: the composition does not decompose by thermal shock and the like while reducing release of any toxic formulation into the environment, can maintain the pipeline's integrity for the extended period by effectively preventing or inhibiting the corrosion or degradation by SRB, and can reduce the costs for excavation of corrosion site, pipeline repair work, coating repair, and frequent examination.

The present invention will be explained below in more detail by way of the examples according to the present invention and comparative examples which are not conducted according to the present invention, but it will be understood by those skilled in the art that the scope of the present invention is not limited by the following examples.

### Experiment Example 1

### Antibacterial Activity Evaluation

Desulfovibrio desulfuricans KCTC 5786 was used as a test sulphate reducing bacteria strain. The medium for culturing the bacteria was Desulfovibrio medium, and the composition of the medium was shown in Table 1:

TABLE 1
Composition of Desulfovibrio medium

| Ingredient | Composition | |
|---|---|---|
| K₂HPO₄ | 0.5 | g |
| NH₄Cl | 1.0 | g |
| Na₂SO₄ | 1.0 | g |
| CaCl₂•2H₂O | 0.1 | g |
| DL-Na-lactate | 2.0 | g |
| Yeast extract | 1.0 | g |
| Resazurin | 1.0 | mg |
| FeSO₄•7H₂O | 0.5 | g |
| Na-thioglycolate | 0.1 | g |
| Ascorbic acid | 0.1 | g |
| Distilled water | 1,000 | ml |

The all materials used in the test, disk paper, medium and so on, were sterilized for 15 min at 121° C. Bacteria's culture and antibacterial activity test were performed in an anaerobic chamber (Anaerobic System, Form a Sci; condition maintaining not more than 5 ppm of oxygen concentration).

After culturing the test strain for not less than 3 days, the volume of the culture was adjusted to 10⁵⁻⁷/mL to prepare for plating it on Desulfovibrio medium, and the test compounds were resolved in a suitable solvent for use (ethanol for lipid soluble compounds, and distilled water for water soluble compounds).

The experiment proceeded with varying the concentration of the compounds used in the experiment to 1.0%, 0.1%, 0.01%, and 0.001%. The 8 mm of a paper disk was placed on the plate prepared by plating the culture, and were inoculated with 50 μl of various concentrations of the compound. After culturing the strain for 24, and 48 hrs at 37° C., the clear zone (mm) representing antibacterial activity was measured and Minimum Inhibitory Concentration (MIC) was determined.

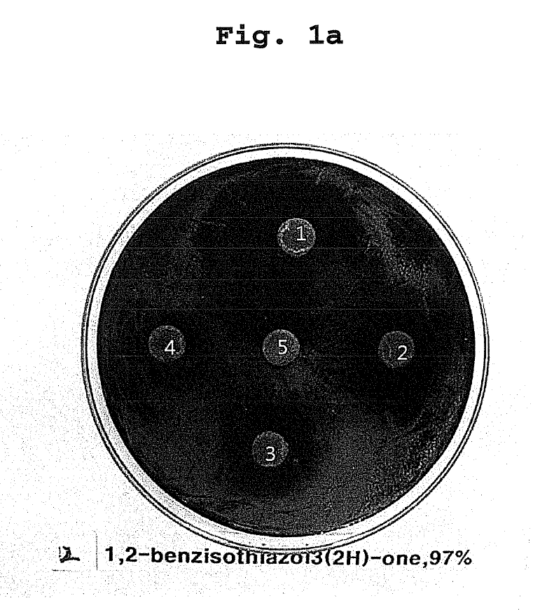

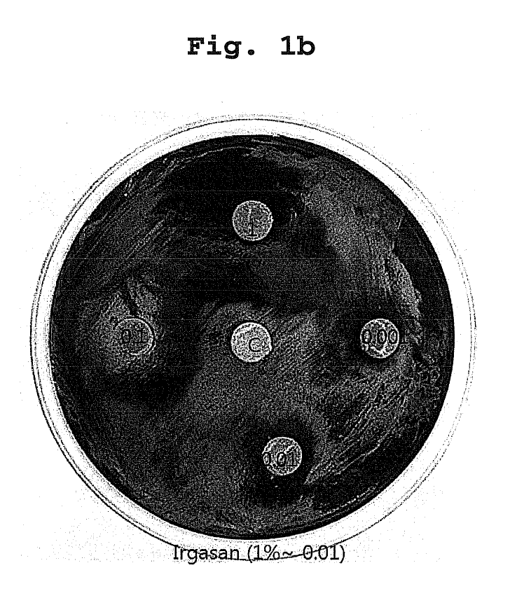

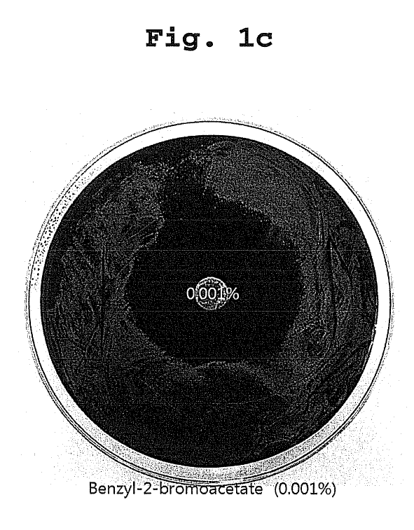

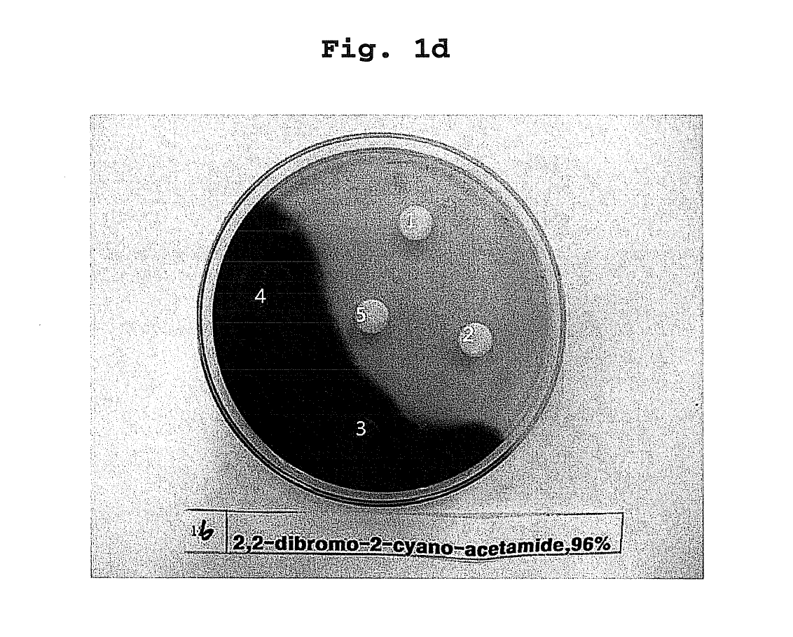

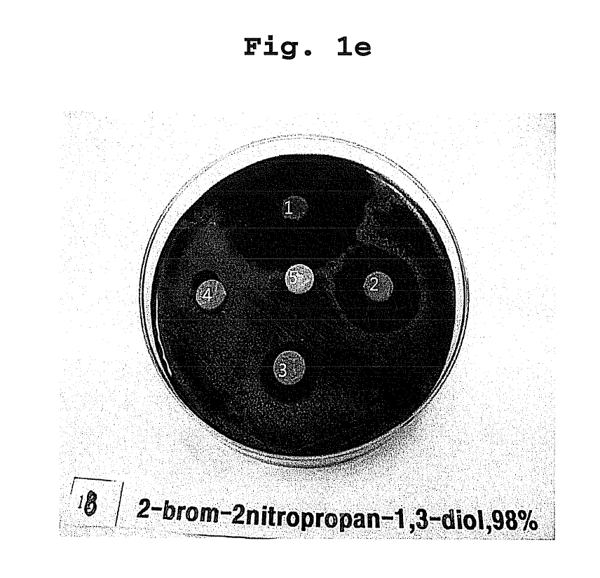

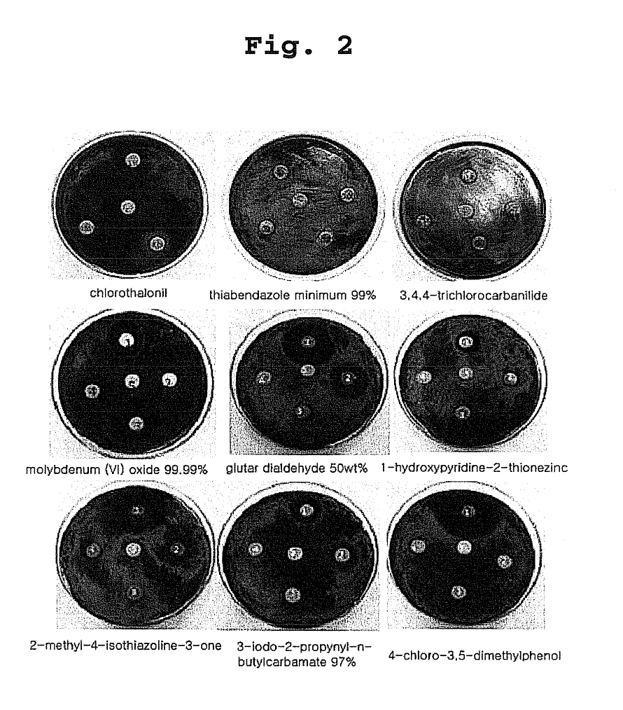

The effective ingredients of the present invention, 1,2-benzisothiazol-3(2H)-one, irgasan, benzyl-2-bromoacetate, 2,2-dibromo-2-cyanoacetamide, and 2-bromo-2-nitropropan-1,3-diol, were shown to have a superior antibacterial activity as MIC of less than 0.01%, and shown to be excellent 10 times more than other antibacterial agents (1-hydroxypyridine-2-tionzinc, 2-methyl-isothiazolin-3-one, and the like) that have been commercially used (see, Table 2, FIGS. 1a to 1e and FIG. 2).

TABLE 2
Test results of antibacterial activity

| Anti- | Antibacterial agent concentration (%) | | | | | | | | |
|---|---|---|---|---|---|---|---|---|---|
| bacterial | 1% | | 0.1% | | 0.01% | | 0.001% | | |
| agent | CZ | | CZ | | CZ | | CZ | | |
| type | (mm) | CZ* | (mm) | CZ* | (mm) | CZ* | (mm) | CZ* | MIC |
| 1,2-benzisothiazol- | 13.5 | ∘ | 6.5 | ∘ | 2.5 | ∘ | 1 | ∘ | 0.001% |
| 3(2H)-one, | | | | | | | | | |
| 97% | | | | | | | | | |
| Irgasan | 14 | ∘ | 11 | ∘ | 5 | ∘ | — | — | 0.01% |
| Benzyl-2- | ≧39 | ∘ | ≧39 | ∘ | ≧39 | ∘ | 35 | ∘ | 0.001% |
| bromoacetate | | | | | | | | | |
| 2,2-dibromo- | ≧39 | ∘ | ≧39 | ∘ | ≧39 | ∘ | ≧39 | ∘ | 0.001% |
| 2-cyano- | | | | | | | | | or less |
| acetamide, | | | | | | | | | |
| 96% | | | | | | | | | |
| 2-bromo-2- | 18.5 | ∘ | 8 | ∘ | 1.5 | ∘ | — | — | 0.01% |
| nitropropan- | | | | | | | | | |
| 1,3-diol, | | | | | | | | | |
| 98% | | | | | | | | | |
| Chloro- | 4 | ∘ | 2 | ∘ | — | — | — | — | 0.1% |
| thalonil | | | | | | | | | |
| Thiabendazole | — | — | — | — | — | — | — | — | 1% |
| minimum | | | | | | | | | or more |
| 99% | | | | | | | | | |
| 3,4,4- | — | — | — | — | — | — | — | — | 1% |
| trichloro- | | | | | | | | | or more |
| Carbanilide | | | | | | | | | |
| Molybdenum | 6 | ∘ | — | — | — | — | — | — | 1.0% |
| (VI) oxide | | | | | | | | | |
| 99.99% | | | | | | | | | |
| Glutar-di- | 12 | ∘ | 6 | ∘ | — | — | — | — | 0.1% |
| aldehyde, | | | | | | | | | |
| 50 wt % | | | | | | | | | |
| 1-hydroxy- | 3.5 | ∘ | 1 | ∘ | — | — | — | — | 0.1% |
| pyridine-2- | | | | | | | | | |
| thionezinc | | | | | | | | | |
| 2-methyl-4- | 12 | ∘ | 5 | ∘ | — | — | — | — | 0.1% |
| isothia- | | | | | | | | | |
| zoline-3-one | | | | | | | | | |
| 3-iodo-2- | 5.5 | ∘ | 3 | ∘ | — | — | — | — | 0.1% |
| propynyl | | | | | | | | | |
| n-butyl- | | | | | | | | | |
| carbamate, | | | | | | | | | |
| 97% | | | | | | | | | |
| 4-chloro- | 16 | ∘ | 1 | ∘ | — | — | — | — | 0.1% |
| 3,5-dimethyl- | | | | | | | | | |
| phenol | | | | | | | | | |

CZ*: whether CZ forms;
CZ: clear zone;
MIC: minimum inhibitory concentration
−: negative.

### Experiment Example 2

### Antibacterial Activity Test after Thermal Shock

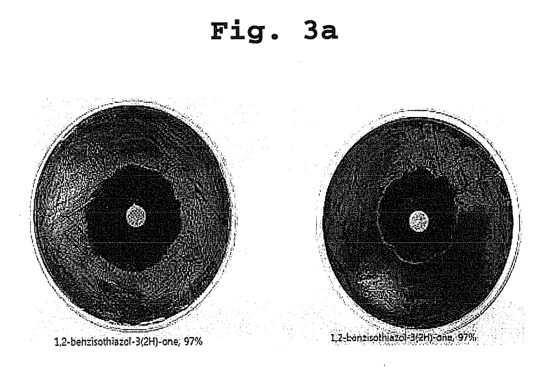

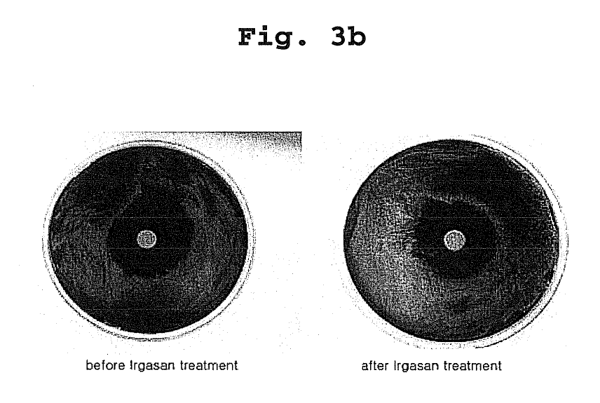

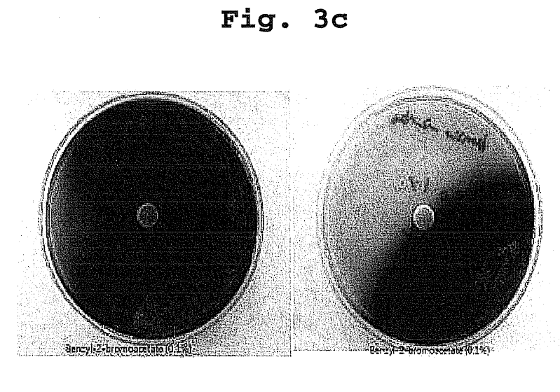

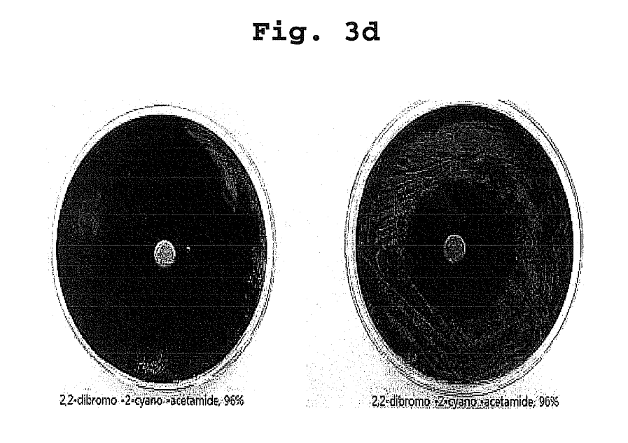

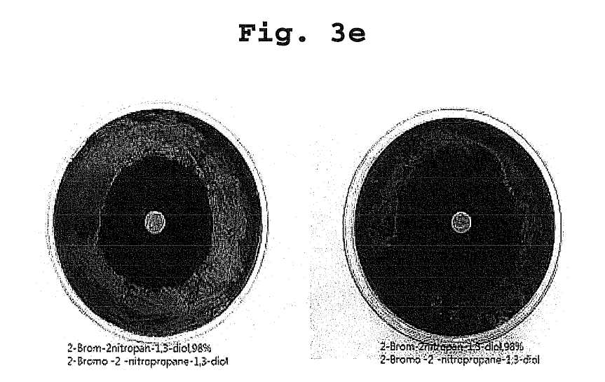

Applying the heat shrinkable sheet using flames of torch, the temperature increased to about 150° C. and the exposure time was around 15 minutes. We would confirm that the organic antibacterial agent shows still the antibacterial activity even after being exposed to such temperature. After 1,2-benzisothiazol-3(2H)-one, irgasan, benzyl-2-bromoacetate, 2,2-dibromo-2-cyanoacetamide, and 2-bromo-2-nitropropan-1,3-diol were exposed to higher temperature (180° C.) for a longer time (1 hour), the antibacterial activity was measured at a concentration of 0.1% using the same method as Experiment Example 1 (see, Table 3, FIGS. 3a to 3e).

As a result, it was found that even after thermal shock, 1,2-benzisothiazol-3(2H)-one, irgasan, benzyl-2-bromoacetate, 2,2-dibromo-2-cyanoacetamide, and 2-bromo-2-nitropropan-1,3-diol maintained the excellent antibacterial activity, and the size of clear zone after thermal shock is was not almost different from the size thereof before thermal shock as can be seen in Table 3.

TABLE 3
Test results of antibacterial activity of 1,2-benzisothiazol-3(2H)-
one, irgasan, benzyl-2-bromoacetate, 2,2-dibromo-2-cyanoacetamide,
and 2-bromo-2-nitropropan-1,3-diol after thermal shock

| | Before thermal | After thermal |
|---|---|---|
| | shock | shock(180° C., 1 hr) |
| Antibacterial agent | clear zone (mm/0.1% condition) | |
| 1,2-benzisothiazol-3(2H)-one | 16 | 14.5 |
| Irgasan | 13 | 13 |
| Benzyl-2-bromoacetate | 39 | 36 |
| 2,2-dibromo-2-cyanoacetamide | 39.5 | 18.5 |
| 2-bromo-2-nitropropan-1,3- | 20 | 25 |
| diol | | |

### Experiment Example 3

### Antibacterial Activity Test of Antibacterial Agent-Added Coating Material

Test equipment and materials, and test condition are as follows:
Coating material type: adhesive (Canusa), primer (Polyken)
Mixed antibacterial agent's concentration: control, 0.5, 1.0, 2.0, 5.0 wt %

As an adhesive specimen, the components were mixed by manual stirring in an oven at 150° C., and then an 1 mm-thick adhesive sheet was fabricated. As a primer specimen, a 0.2 mm-thick sheet having a primer dry film was fabricated. The specimen was diced into a size of 15 mm×15 mm, and after UV sterilization, the antibacterial activity of the specimen was assessed using the same environment and method as in the test method of Experiment Example 1 described above.

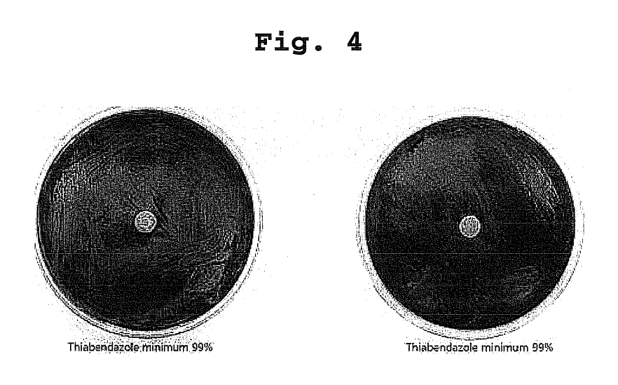

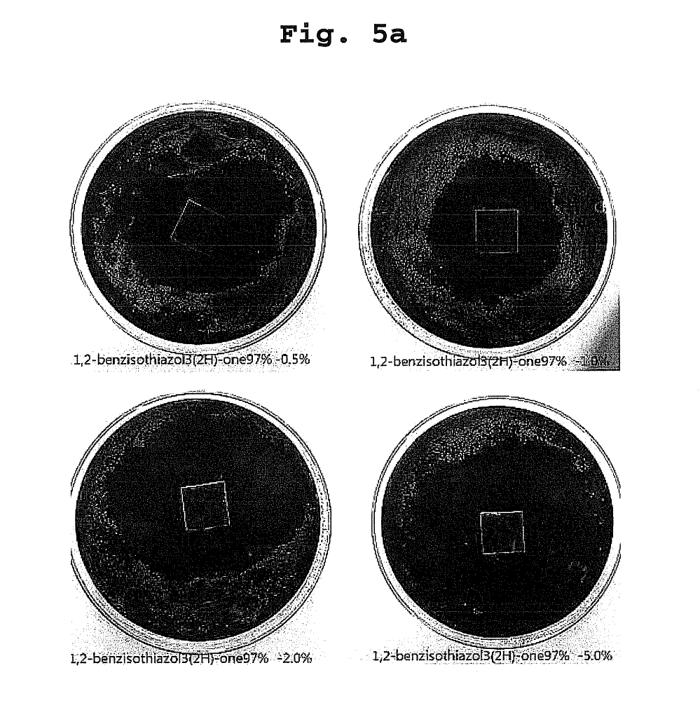

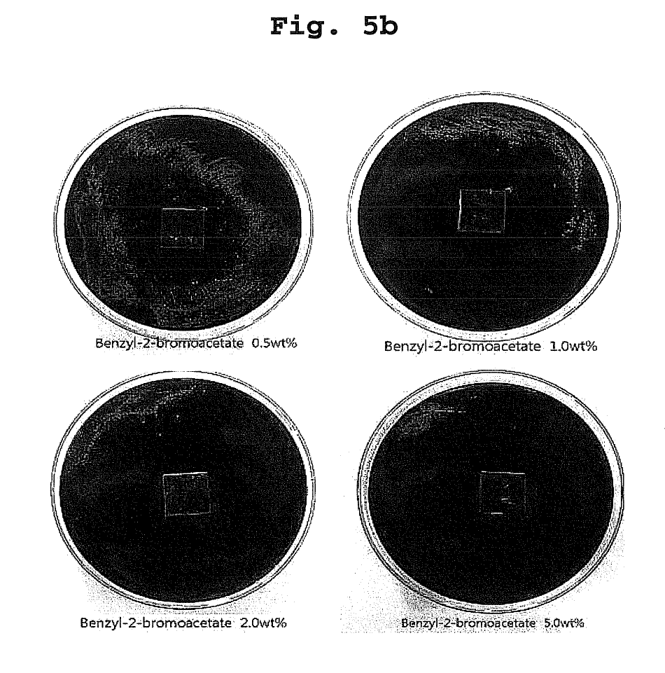

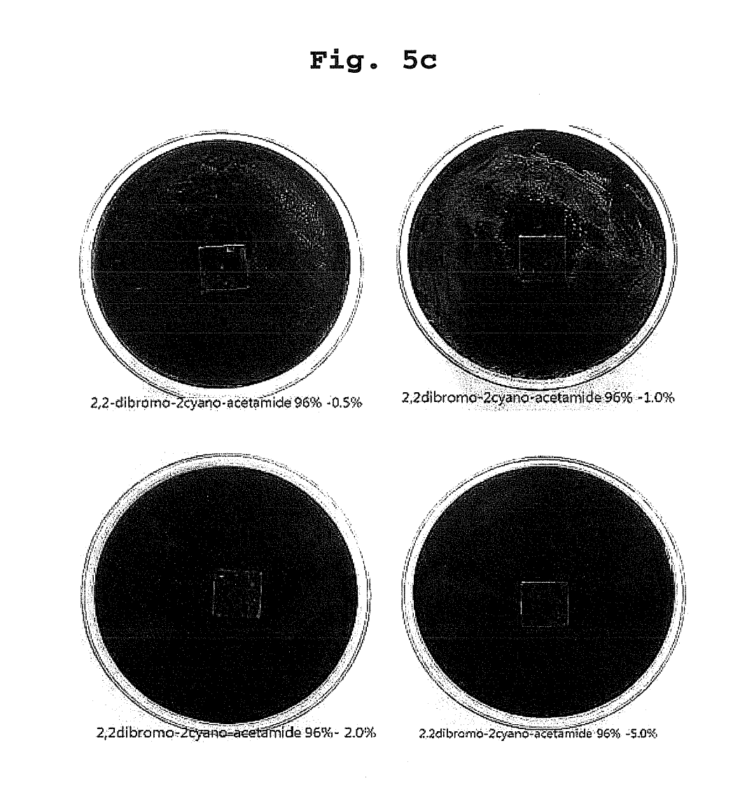

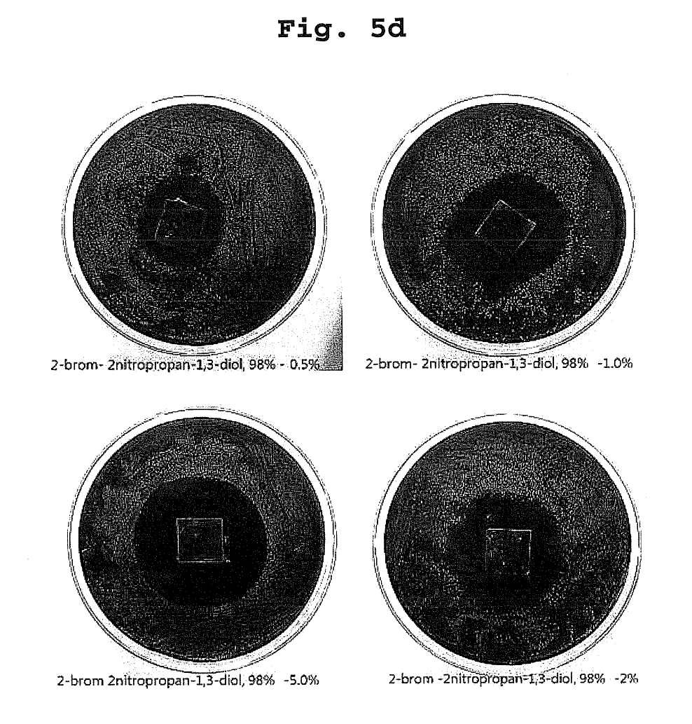

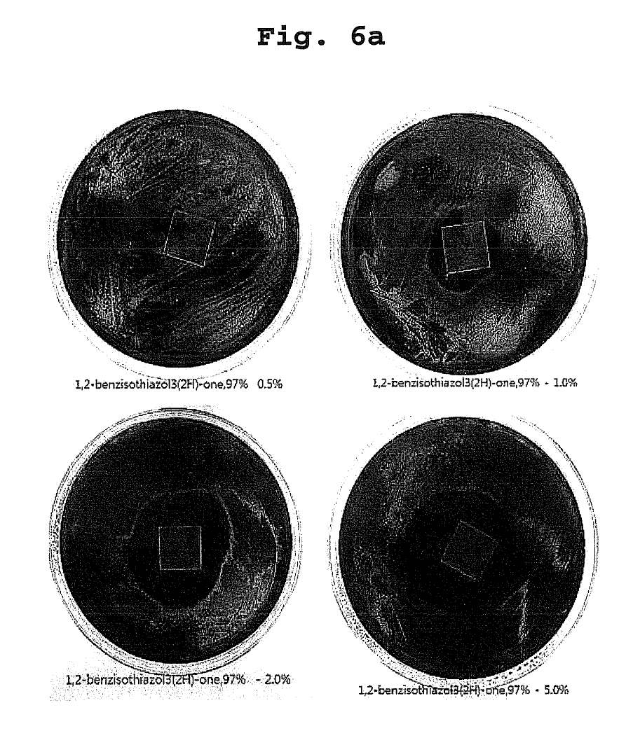

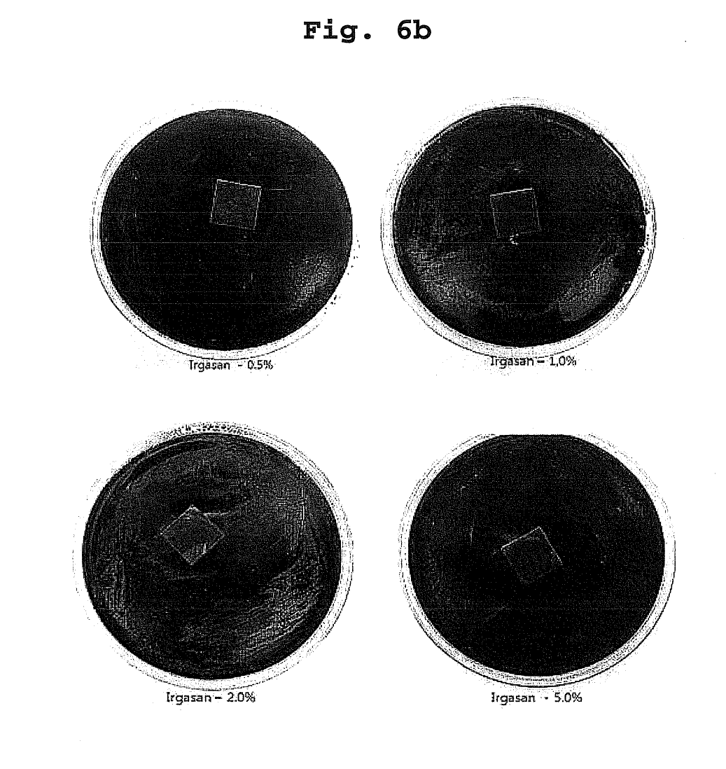

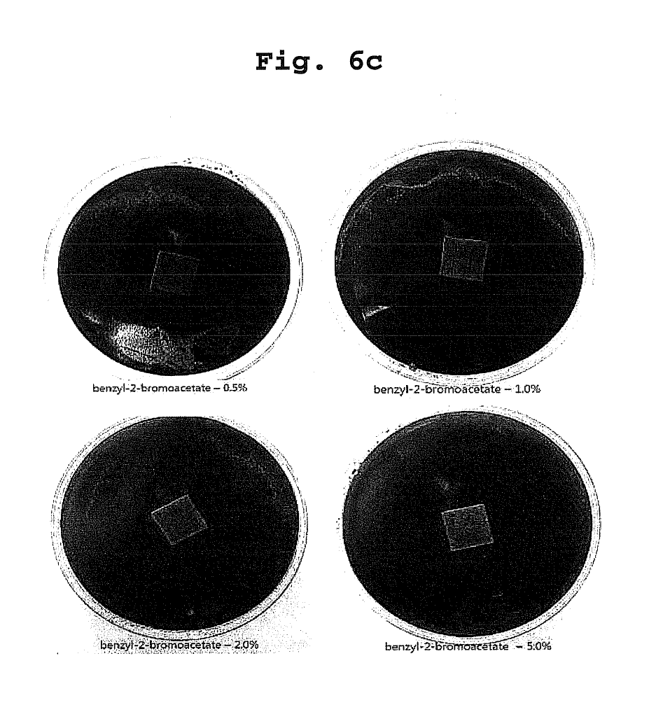

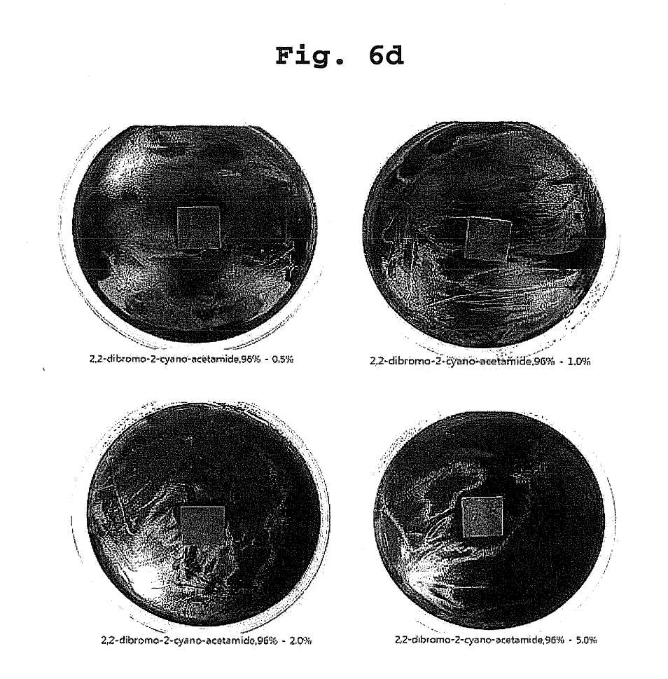

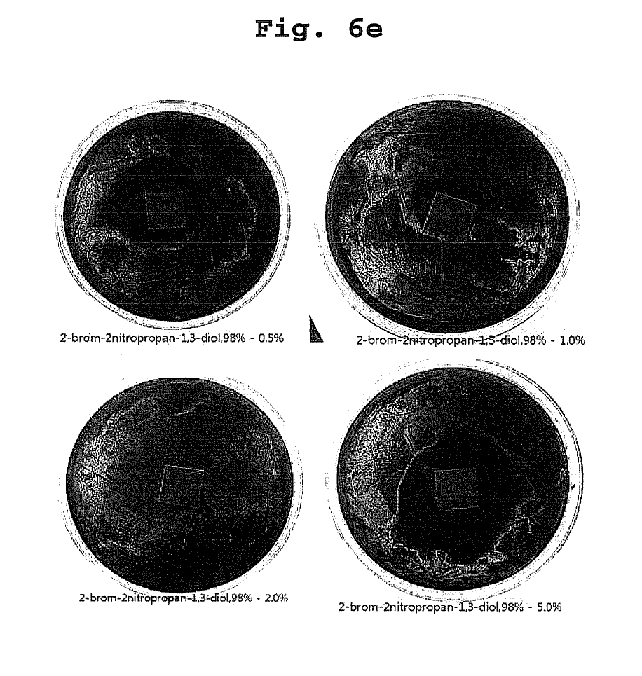

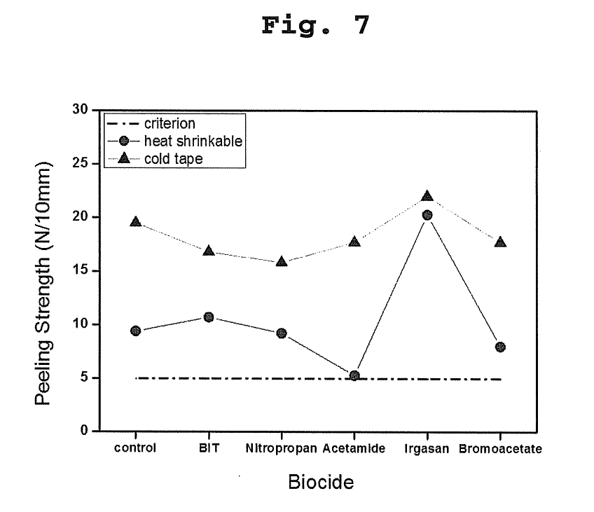

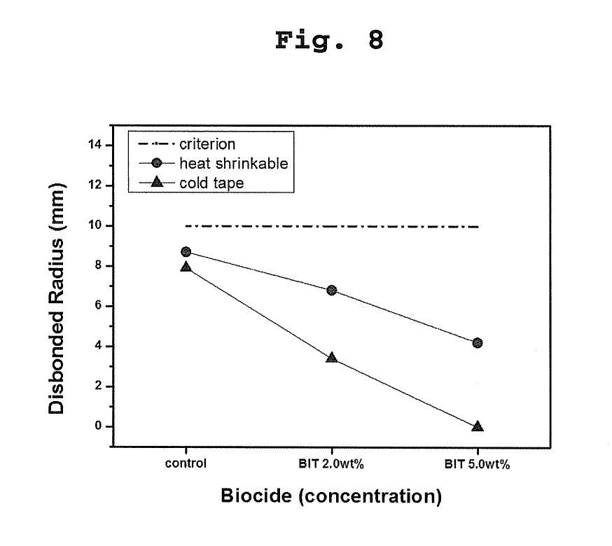

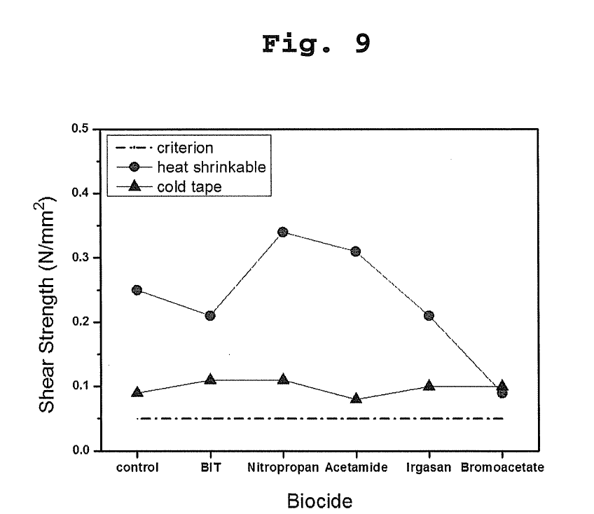

According to the experiment results, the antibacterial activity was observed in the adhesive regardless of the added antibacterial agent's concentration. In case that the antibacterial agent was added to the primer, although some irregular tendency was shown presumably due to non-uniform mixing of the antibacterial agent even when the added antibacterial agent's concentration increased, the specimen demonstrated the antibacterial activity (see, Table 4, Table 5, FIG. 4, FIGS. 5a to 5d).

TABLE 4
Test results for antibacterial activity
of irgasan-added coating material

| | Clear zone(mm) | | | | |
|---|---|---|---|---|---|
| Antibacterial | Primer | | | | |
| agent | control | 0.5 wt % | 1.0 wt % | 2.0 wt % | 5.0 wt % |
| Irgasan | — | 3.5 | 1.5 | 1.5 | 6 |

TABLE 5
Test results for antibacterial activity of
other antibacterial agent-added coating material

| | Clear zone (mm) | | | | | | | | | |
|---|---|---|---|---|---|---|---|---|---|---|
| Antibacterial | Adhesive (wt %) | | | | | Primer (wt %) | | | | |
| agent | Control | 0.5 | 1.0 | 2.0 | 5.0 | control | 0.5 | 1.0 | 2.0 | 5.0 |
| 1,2-benzisothiazol- | — | 15 | 18 | 19 | 25 | — | — | 7.5 | 8.5 | 17.5 |
| 3(2H)- | | | | | | | | | | |
| one | | | | | | | | | | |
| Benzyl-2- | — | 24 | 30 | 34 | ≧39 | — | ≧26 | ≧32 | ≧34 | ≧39 |
| bromo-acetate | | | | | | | | | | |
| 2,2-dibromo-2- | — | 2 | 2.5 | ≧26 | ≧30.5 | — | 1.5 | — | 1 | 6.5 |
| cyanoacetamide | | | | | | | | | | |
| 2-bromo-2- | — | 5.5 | 10.5 | 13 | 14.5 | — | 9 | 9 | 2 | 16.5 |
| nitropropan- | | | | | | | | | | |
| 1,3-diol | | | | | | | | | | |

### Experiment Example 4

### Assessment of Coating Physical Properties

A. Preparation of Coating Material for Adhesive Strength Test

For an experimental heat shrinkable sheet material, only the commercial Canusa WLS adhesive out of commercial heat shrinkable sheet (Canusa WLS) was taken at low temperature, and 5 wt % of antibacterial agent was added to the adhesive and then exposed to in an oven at 150° C. for a certain period of time to induce a flowability fluidity. To mix them more uniformly, they were sufficiently mixed by a stirrer while maintaining the above temperature on a heating mantle, and antibacterial agent-added adhesive sheets of 100 (W)×400 (L)×1.2 (T) were fabricated. The outside backing (thermal contraction heat shrinkable crosslinked polyethylene) was prepared by cutting the commercial product produced by Koschem (Republic of Korea) into the above size. Thereafter, the experimental thermal contraction heat shrinkable sheet composed of sets of the antibacterial agent-added adhesive sheet and the crosslinked polyethylene was fabricated. Dusts from sand blasting (surface treatment grade SSPC10)-treated 4″ pipeline having a length of 10 cm were removed from sand blasting (surface treatment grade SSPC10)-treated 4″ pipeline having a length of 10 cm, and then, lipids residual oil were also removed using acetone. After pre-heating the pipeline to a temperature of 60° C. as in the requirements for commercial products, the fabricated experimental thermal contraction heat shrinkable sheet was applied thereon while heating using a torch.

For an experimental tape, to the primer produced by Polyken was mixed 5 wt % of the antibacterial agent based on the dry film weight. Dusts from sand blasting (surface treatment grade SSPC10)-treated 4″ pipeline having a length of 10 cm were removed from sand blasting (surface treatment grade SSPC10)-treated 4″ pipeline having a length of 10 cm, and then, lipids residual oil were also removed using acetone. Thereafter, the pipeline was pre-heated to a temperature of 40° C. The prepared primer was applied to the pipeline and the pipeline was rolled up in a tape (Polyken).

B. Adhesive Strength Test

The coating material applied to the pipeline was diced into three strips having a length of 20 cm and a width of 1 cm. The strip was placed over a universal testing machine (Instron 4467) and pulled at a speed of 10 mm/min. For each of three results, twenty data were taken at regular intervals and the average values for the data were calculated. Thereafter, the average value for all of three specimens was calculated.

C. Shear Strength

The adhesive sheet prepared as in “A” was cut into a size of 2 cm×5 cm. Sand basted two steel plates treated with sand blasting (surface treatment grade SSPC10) having a size of 5 cm×10 cm, were overlapped partially in an area of 2 cm×5 cm, and the adhesive was bonded only to two overlapped areas while applying sufficient heat. Likewise, for testing the primer and tape, the antibacterial agent-added primer and inner layer were bonded to the overlapped areas of 2 cm×5 cm. Each of five specimens was placed over the universal testing machine and pulled at a speed of 10 mm/min. Mean value for maximal values was calculated.

D. Cathodic Disbondment Resistance

To the coating specimen prepared as in “A” was made an artificial defect having a diameter of 6 mm and an acrylic cell was attached to a surrounding region of the defect. After filling 0.5 M of a NaCl solution into the cell, a voltage of −1.5 V was applied on the basis of saturated copper sulphate electrode using a potentiostat for 28 days. Thereafter, the radius peeled disbonded from the artificial defect was measured.

According to the experiment results for such adhesive strength, cathodic disbondment resistance and shear strength, the adhesive strength, shear strength, cathodic disbondment resistance and the like of coating materials comprising 1,2-benzisothiazol-3(2H)-one, irgasan, benzyl-2-bromoacetate, 2,2-dibromo-2-cyanoacetamide, and 2-bromo-2-nitropropan-1,3-diol were confirmed to have the values above levels required in EN12068 European standard EN12068.

As discussed above, the composition of the present invention has the advantage as follows: while reducing release of any toxic formulation into the environment, the composition does not decompose by thermal shock and the like, can maintain the pipeline's soundness integrity for the extended period by effectively preventing or inhibiting the corrosion or degradation by SRB, and can reduce the costs for excavation of corrosion site, pipeline repair work, coating repair, and frequent examination.

While the invention has been shown and described with respect to the particular embodiments, it will be understood by those skilled in the art that various changes and modification may be made.

What is claimed is:

1. A composition for inhibiting proliferation of sulphate reducing bacteria, the composition comprising at least one of 1,2-benzisothiazol-3(2H)-one, irgasan, benzyl-2-bromoacetate, 2,2-dibromo-2-cyanoacetamide, and 2-bromo-2-nitropropan-1,3-diol as effective ingredients.

2. The composition of claim 1, further comprising a binder.

3. A method for inhibiting proliferation of sulphate reducing bacteria, the method comprising the step of including a sufficient amount for inhibiting the proliferation of sulphate reducing bacteria of at least one of 1,2-benzisothiazol-3(2H)-one, irgasan, benzyl-2-bromoacetate, 2,2-dibromo-2-cyanoacetamide, and 2-bromo-2-nitropropan-1,3-diol in a corrosion sensitive material or degradation sensitive material.

4. The method of claim 3, wherein the corrosion sensitive material is a metal.

5. The method of claim 4, wherein the metal is selected from the group consisting of carbon steel, stainless steel, aluminum, aluminum alloy, copper, copper alloy, titanium, titanium alloy, nickel and nickel alloy.

6. The method of claim 3, wherein the degradation sensitive material is selected from the group consisting of concrete, reinforced concrete and cement.

7. A sheet comprising a composition for inhibiting proliferation of sulphate reducing bacteria comprising at least one of 1,2-benzisothiazol-3(2H)-one, irgasan, benzyl-2-bromoacetate, 2,2-dibromo-2-cyanoacetamide, and 2-bromo-2-nitropropan-1,3-diol.

8. The sheet of claim 7, wherein the sheet is applied to the corrosion sensitive material or degradation sensitive material.

9. The sheet of claim 7, wherein the sheet is at least one sheet selected from the group consisting of a heat shrinkable sheet, a adhesive sheet, an insulating polymer sheet, and a plastic sheet.

10. A steel plate to which a composition for inhibiting proliferation of sulphate reducing bacteria comprising at least one of 1,2-benzisothiazol-3(2H)-one, irgasan, benzyl-2-bromoacetate, 2,2-dibromo-2-cyanoacetamide, and 2-bromo-2-nitropropan-1,3-diol is applied.
